(19) United States
(12) Patent Application Publication
(10) Pub. No.: US 2014/0336499 A1
(43) Pub. Date: Nov. 13, 2014

(54) METHOD FOR CREATING A DETAILED ATTENUATION VALUE MAP FOR A LIMITED BODY REGION

(71) Applicant: Siemens Aktiengesellschaft, Munich (DE)

(72) Inventors: Matthias FENCHEL, Erlangen (DE); Björn HEISMANN, Erlangen (DE); Kirstin JATTKE, Nuremberg (DE); Ralf LADEBECK, Erlangen (DE); Sebastian SCHMIDT, Weisendorf (DE)

(73) Assignee: SIEMENS AKTIENGESELLSCHAFT, Munich (DE)

(21) Appl. No.: 14/267,995

(22) Filed: May 2, 2014

(30) Foreign Application Priority Data
May 8, 2013 (DE) 102013208500.1

(51) Int. Cl. A61B 6/00; G01R 33/48; A61B 6/03

(52) U.S. Cl. CPC A61B 6/5211; A61B 6/037; G01R 33/481
USPC 600/411; 600/425

(57) ABSTRACT

A method, medical imaging device and computer program product are disclosed for creating a detailed attenuation value map for a limited body region of a patient for a positron emission tomography examination. In an embodiment, the method includes an acquisition of first attenuation value data from a first attenuation value measurement of the limited body region; an ascertainment of at least one mean attenuation value based upon the first attenuation value data for the limited body region; an acquisition of second attenuation value data from a second attenuation value measurement of the limited body region; an ascertainment of local correction values based upon the second attenuation value data for the limited body region; and a determination of the detailed attenuation value map for the limited body region based upon the at least one mean attenuation value and based upon the local correction values.

### PRIORITY STATEMENT

The present application hereby claims priority under 35 U.S.C. §119 to German patent application number DE 102013208500.1 filed May 8, 2013, the entire contents of which are hereby incorporated herein by reference.

### FIELD

At least one embodiment of the present invention is based on a method for creating a detailed attenuation value map for a limited body region of a patient for a positron emission tomography examination via a medical imaging device. At least one embodiment of the present invention is furthermore based on a medical imaging device which comprises a positron emission tomography unit, a magnetic resonance imaging device and/or a system control unit and which is designed in order to execute a method for creating a detailed attenuation value map for a limited body region of a patient for a positron emission tomography examination. At least one embodiment of the present invention moreover comprises a computer program product containing an executable program which when installed on a computer executes a method for creating a detailed attenuation value map for a limited body region of a patient for a positron emission tomography examination by way of a medical imaging device.

### BACKGROUND

For combined magnetic resonance positron emission tomography examinations, an attenuation value map is created prior to a positron emission tomography measurement. To this end, a tissue-dependent attenuation correction is ascertained by way of a magnetic resonance measurement, in which case a segmentation of an examination region takes place and averaged attenuation values for the individual segments of the segmentation are ascertained from the magnetic resonance measurement.

Individual organs and/or body regions, in particular a lung region, of a patient do however exhibit a greater scattering and/or variance in respect of their attenuation properties than other body regions of the patient, which means that an averaged attenuation value for the lung only corresponds imprecisely to an actual attenuation value. In particular during an acquisition and/or a quantification of lesions in the thorax and/or in a lung region of a patient this can result in undesired inaccuracies. Furthermore, pathological changes within the lung region frequently lead to a change in the density of a lung, such as for example in the case of emphysema and/or in the case of atelectasis etc., which have a direct effect on an attenuation of photons as they pass through the lung.

### SUMMARY

At least one embodiment of the present invention is directed to making available a detailed attenuation value map for, in particular, inhomogeneous body regions of the patient for a positron emission tomography examination. Advantageous embodiments are described in the subclaims.

At least one embodiment of the invention is based on a method for creating a detailed attenuation value map for a limited body region of a patient for a positron emission tomography examination by means of a medical imaging device, comprising:
an acquisition of first attenuation value data from a first attenuation value measurement of the limited body region via the medical imaging device,
an ascertainment of at least one mean attenuation value on the basis of the first attenuation value data for the limited body region,
an acquisition of second attenuation value data from a second attenuation value measurement of the limited body region via the medical imaging device,
an ascertainment of local correction values on the basis of the second attenuation value data for the limited body region and
a determination of the detailed attenuation value map for the limited body region on the basis of the at least one mean attenuation value and on the basis of the local correction values.

By preference, at least one embodiment of the medical imaging device comprises a magnetic resonance imaging device. By way of the first attenuation value data, essentially a tissue structure of different limited body regions is acquired and a mean attenuation value is ascertained and/or determined on the basis of said tissue structure. In such a manner a consistent mean attenuation value can be present for example for individual body regions, although said body regions are particularly inhomogeneous in respect of material and/or matter distribution, such as for example the lung region of the patient. The mean attenuation value can be ascertained with the aid of values stored in a database and/or with the aid of values from a known computed tomography measurement, in which case the mean attenuation value essentially comprises an average attenuation value for the limited body region. By means of the second attenuation value data on the other hand, essentially a correction profile of the limited body region can be ascertained and/or created and in such a manner the mean attenuation value can be adjusted and/or corrected.

Furthermore, at least one embodiment of the invention is based on a medical imaging device which comprises a positron emission tomography unit, a magnetic resonance imaging device and a system control unit and which is designed in order to execute a method for creating a detailed attenuation value map for a limited body region of a patient for a positron emission tomography examination having at least the following steps:
an acquisition of first attenuation value data from a first attenuation value measurement of the limited body region by means of the magnetic resonance imaging device,
an ascertainment of at least one mean attenuation value on the basis of the first attenuation value data for the limited body region by means of the system control unit,
an acquisition of second attenuation value data from a second attenuation value measurement of the limited body region by means of the magnetic resonance imaging device,
an ascertainment of local correction values for the limited body region on the basis of the second attenuation value data by means of the system control unit and
a determination of the detailed attenuation value map for the limited body region on the basis of the at least one mean attenuation value and the local correction values by means of the system control unit.

Furthermore, at least one embodiment of the invention is based on a computer program product which comprises a program and can be loaded directly in a storage unit of a programmable system control unit of a medical imaging device, having program segments/modules in order to execute a method according to at least one embodiment of the invention when the program is executed in the system control unit of the medical imaging device. With the computer program product, all or different forms of embodiment described above of the method according to at least one embodiment of the invention can be executed when the computer program product is executed in the system control unit of the medical imaging device. In this situation the computer program product can utilize additional program segments/modules, such as for example libraries and auxiliary functions, in order to implement an appropriate form of embodiment of the method.

Further advantages, features and details of the invention will emerge from the example embodiment described in the following and with reference to the drawings.

### BRIEF DESCRIPTION OF THE DRAWINGS

In the drawings:

FIG. 1 shows a method according to an embodiment of the invention for creating a detailed attenuation value map for a limited body region and

FIG. 2 shows a medical imaging device for executing the method illustrated in FIG. 1.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention is based on a method for creating a detailed attenuation value map for a limited body region of a patient for a positron emission tomography examination by means of a medical imaging device, comprising:
an acquisition of first attenuation value data from a first attenuation value measurement of the limited body region via the medical imaging device,
an ascertainment of at least one mean attenuation value on the basis of the first attenuation value data for the limited body region,
an acquisition of second attenuation value data from a second attenuation value measurement of the limited body region via the medical imaging device,
an ascertainment of local correction values on the basis of the second attenuation value data for the limited body region and
a determination of the detailed attenuation value map for the limited body region on the basis of the at least one mean attenuation value and on the basis of the local correction values.

As a result of this embodiment of the invention, it is possible to create a particularly detailed attenuation value map for the limited body region, for example for a lung region of the patient which is formed inhomogeneously in respect of its tissue properties and/or its material properties, since different subregions of the limited body region having different local correction values preferably also have different attenuation properties for positron emission tomography signals. In such a manner the at least one mean attenuation value can be adjusted and/or corrected locally on the basis of the local correction values, in particular for individual subregions of the lung, in which case the different subregions can have a common mean attenuation value. It is furthermore possible by means of the detailed attenuation value map to make available as exact an attenuation value map as possible in particular in the region of the thorax of the patient, which means that an unambiguous identification of lesions in the region of the thorax is enabled. In particular, a quantitative measurement of a radiotracer image within a lesion (SUV values) can be enhanced here on account of an improved accuracy of the attenuation value map because errors in the attenuation value map have hitherto resulted in an overestimate or an underestimate. This is particularly problematical in a lung region because here the attenuation varies widely on account of varying proportions of air, tissue and blood, which means that the method according to the invention can be employed to particular benefit for positron emission tomography examination in a lung region of the patient.

By preference, at least one embodiment of the medical imaging device comprises a magnetic resonance imaging device. By way of the first attenuation value data, essentially a tissue structure of different limited body regions is acquired and a mean attenuation value is ascertained and/or determined on the basis of said tissue structure. In such a manner a consistent mean attenuation value can be present for example for individual body regions, although said body regions are particularly inhomogeneous in respect of material and/or matter distribution, such as for example the lung region of the patient. The mean attenuation value can be ascertained with the aid of values stored in a database and/or with the aid of values from a known computed tomography measurement, in which case the mean attenuation value essentially comprises an average attenuation value for the limited body region. By means of the second attenuation value data on the other hand, essentially a correction profile of the limited body region can be ascertained and/or created and in such a manner the mean attenuation value can be adjusted and/or corrected.

Particularly advantageously, the acquisition of the second attenuation value data takes place on the basis of a proton density weighted attenuation value measurement, in which case the local correction values comprise local density values. By way of the local density values it is advantageously possible to ascertain a density, in particular a density distribution and/or a density profile, of the limited body region, in particular of a lung region, of the patient and in such a manner to ascertain a local material property and/or an attenuation property of the limited body region. In particular, on the basis of the local density values and/or a local material property ascertained therefrom the mean attenuation value can be adjusted and/or corrected locally, in particular for individual subregions and/or individual segments of the limited body region.

Particularly advantageously, the limited body region of the patient comprises an organ region, in particular a lung region, of the patient. In such a manner it is possible in particular for the lung region, which exhibits an inhomogeneous and/or temporally variable density distribution, to create as exact an attenuation value map as possible and thereby to advantageously increase the reliability and/or precision of a positron emission tomography examination of the lung region.

In an advantageous development of at least one embodiment of the invention it is proposed that on the basis of the second attenuation value data a segmentation of the limited body region takes place and a local correction value is ascertained for each segment of the limited body region on the basis of the second attenuation value data. In such a manner it is possible to make available a detailed attenuation value map having a particularly high spatial resolution for a positron emission tomography examination. In this context a segmentation of the limited body region is understood in particular to be a subdivision of the limited body region into individual subregions, in which case the subdivision can take place on the basis of a size of the subregions and/or on the basis of a matter property and/or a homogeneity of the subregions and/or on the basis of further criteria appearing meaningful to the person skilled in the art. In particular, the segmentation of the limited body region comprises a subdivision of body regions provided with a consistent mean attenuation value into at least two or more individual segments and/or into at least two or more individual subregions. Provided that a segmentation of the limited body region has likewise taken place on the basis of the first attenuation value data, the segmentation of the limited body region on the basis of the second attenuation value data exhibits a higher spatial resolution than the segmentation on the basis of the first attenuation value data.

It is furthermore proposed that image data is reconstructed on the basis of the second attenuation value data and the local correction value is determined at least partially by way of an association of correction values with the reconstructed image data on the basis of a characteristic curve. In such a manner it is possible to determine and/or ascertain the local correction values, in particular the local density values, preferably directly and in time-saving fashion on the basis of the reconstructed image data and the characteristic curve. For example, on the basis of different gray-scale values in the reconstructed image data the association of correction values, in particular density values, can take place on the basis of the characteristic curve. The characteristic curve can advantageously comprise a linear characteristic curve.

It is moreover proposed that at least one parameter and/or at least one information item from the characteristic curve is determined for an association of the second attenuation value data with a local correction value on the basis of a correlating comparison measurement and/or on the basis of a calibration measurement on a further body region, which is different to the limited body region, of the patient. A particularly simple and rapid determination of the characteristic curve can be achieved by this means and an exact and rapid association of local correction values, in particular of local density values, with an acquired attenuation value by way of the characteristic curve can thereby take place. A correlating comparison measurement is for example understood to be a correlating computed tomography measurement (CT measurement), in particular having a suitable normalization, in which case the data from the CT measurement can be stored in a storage unit. Furthermore, a calibration measurement is understood in particular to be a measurement for correction data acquisition, in particular for density data acquisition and/or attenuation value acquisition, which is made on an organ and/or on a body region of the patient having small fluctuations in density and/or having small fluctuations of attenuation values by means of the medical imaging device, in particular the magnetic resonance device, such as for example a correlating magnetic resonance measurement on a liver region of the patient.

In a further embodiment of the invention it is proposed that a gravitational force acting on the limited body region is incorporated into the calculation of the local correction values, by which local disturbances and/or inaccuracies in the local correction values, in particular the local density values, can be reduced and/or minimized. In addition, in such a manner a correction gradient, in particular a density gradient, along the force due to weight can be ascertained and by this means particularly exact local density values of the limited body region can be made available for the determination of the detailed attenuation value map. Furthermore, incomplete measurements, for example a measurement having only one or a few two-dimensional layers, can by this means also be completed in computational terms by means of the density gradient. In such a manner, even with a known density gradient a density profile of the limited body region can be ascertained in order to create the detailed attenuation value map from one or a few two-dimensional layers of a magnetic resonance measurement.

Susceptibility artifacts in the second attenuation value data can advantageously be minimized and/or prevented if the acquisition of the second attenuation value data takes place by means of at least one sequence having a short echo time. In this context a short echo time is understood in particular to be an echo time of less than 500 μs. Short and/or ultrashort echo times are used for example in the case of the UTE sequence and/or in the case of the PETRA sequence and/or further sequences appearing meaningful to the person skilled in the art.

Furthermore, it is proposed that the detailed attenuation value map is created depending on a first position of the limited body region, which means that the detailed attenuation value map can preferably be made available for one measurement position of the limited body region for the positron emission tomography examination (PET examination). For example, a detailed attenuation value map of the lung of the patient can be created during an inhaled state or an exhaled state of the lung, in which case here a positron emission tomography data acquisition can take place as a so-called “gated-PET examination” also only in the exhaled state or in the inhaled state of the patient. By preference, the second attenuation value data from the second attenuation value measurement is acquired depending on the first position of the limited body region.

In an advantageous development of at least one embodiment of the invention it is proposed that the detailed attenuation value map for a second position of the limited body region is calculated on the basis of the second attenuation value data for the first position of the limited body region. In such a manner it is possible with minimal measurement work to make available a detailed attenuation value map for different positions and/or different states of the limited body region of the patient for the positron emission tomography examination. For example, also incorporated into the calculation and/or into an interpolation of the detailed attenuation value map for the second position and/or a second state is a known and/or expected volume change of a lung region having a constant material quantity of tissue, in particular lung tissue, and/or a constant material quantity of bodily fluids in the lung region.

Furthermore, at least one embodiment of the invention is based on a medical imaging device which comprises a positron emission tomography unit, a magnetic resonance imaging device and a system control unit and which is designed in order to execute a method for creating a detailed attenuation value map for a limited body region of a patient for a positron emission tomography examination having at least the following steps:
an acquisition of first attenuation value data from a first attenuation value measurement of the limited body region by means of the magnetic resonance imaging device,
an ascertainment of at least one mean attenuation value on the basis of the first attenuation value data for the limited body region by means of the system control unit,
an acquisition of second attenuation value data from a second attenuation value measurement of the limited body region by means of the magnetic resonance imaging device,
an ascertainment of local correction values for the limited body region on the basis of the second attenuation value data by means of the system control unit and
a determination of the detailed attenuation value map for the limited body region on the basis of the at least one mean attenuation value and the local correction values by means of the system control unit.

Through this embodiment of the invention it is possible to create a particularly detailed attenuation value map for the limited body region, for example for a lung region of the patient formed inhomogeneously in respect of its tissue properties and/or its material properties, because different subregions of the limited body region having different local correction values preferably also exhibit different attenuation properties for positron emission tomography signals. In such a manner it is possible to locally, in particular for individual subregions of the lung, adjust and/or correct the at least one mean attenuation value on the basis of the local correction values, in which case the different subregions can exhibit a common mean attenuation value. In addition, it is possible by means of the detailed attenuation value map to make available as exact an attenuation value map as possible in particular in the region of the thorax of the patient, which means that an unambiguous identification of lesions in the region of the thorax is enabled.

Furthermore, at least one embodiment of the invention is based on a computer program product which comprises a program and can be loaded directly in a storage unit of a programmable system control unit of a medical imaging device, having program segments/modules in order to execute a method according to at least one embodiment of the invention when the program is executed in the system control unit of the medical imaging device. With the computer program product, all or different forms of embodiment described above of the method according to at least one embodiment of the invention can be executed when the computer program product is executed in the system control unit of the medical imaging device. In this situation the computer program product can utilize additional program segments/modules, such as for example libraries and auxiliary functions, in order to implement an appropriate form of embodiment of the method.

Photon pairs are acquired for positron emission tomography examinations (PET examinations) by means of a positron emission tomography unit 120 (PET unit 120), in particular by means of a positron emission tomography detector array 121 (PET detector array 121). The photon pairs result from an annihilation of a positron with an electron, in which case each of the photons has an energy of 511 keV. Trajectories of the two photons in this situation enclose an angle of 180°.

The positron causing the annihilation together with the electron is emitted here by a radiopharmaceutical agent, in which case the radiopharmaceutical agent is administered to a patient 101 by way of an injection. As they pass through matter the photons created during the annihilation can be absorbed and/or attenuated, in which case the absorption probability depends on the path length through the matter and the corresponding absorption coefficient of the matter. Accordingly, when PET signals are evaluated a correction of said PET signals is required by means of an attenuation value map created for this purpose in respect of the attenuation by components which are situated in the beam path.

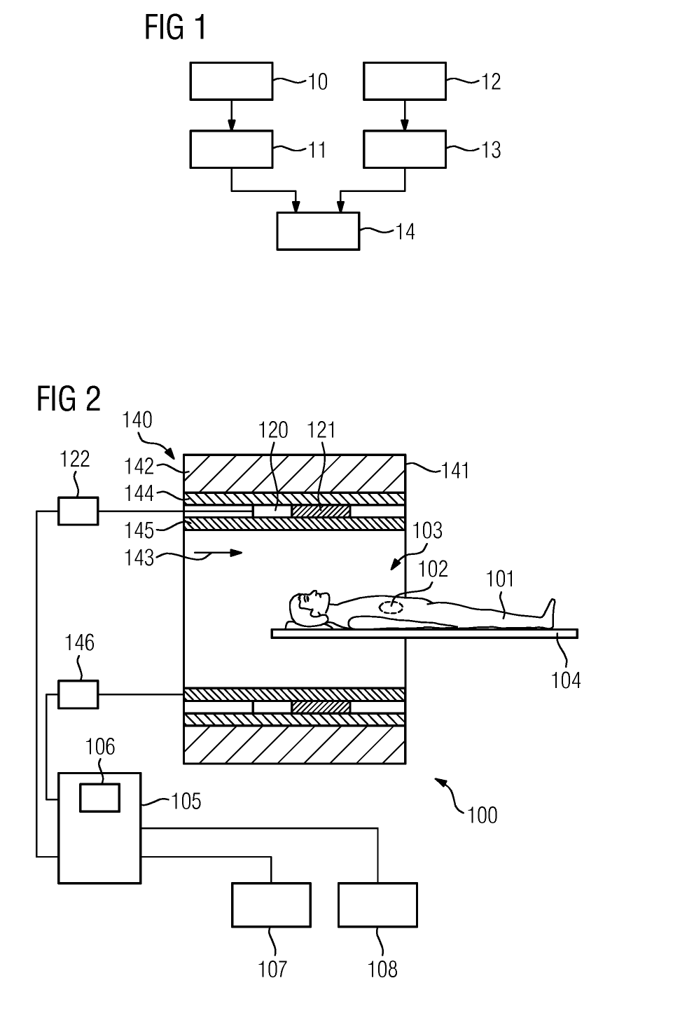

FIG. 1 shows a schematic illustration of a method for creating a detailed attenuation value map for a limited body region 102 of the patient 101 for the PET examination. To this end the patient 101 is firstly introduced into a patient receiving area 103 of a medical imaging device 100 (FIG. 2). The medical imaging device 100 is formed by a combined imaging device which comprises a magnetic resonance imaging device 140 and the PET unit 120.

In order to ascertain and/or create the detailed attenuation value map the patient 101 is firstly positioned on a patient supporting device 104 of the medical imaging device 100. The patient supporting device 104 together with the patient 101 is positioned inside the patient receiving area 103 of the medical imaging device 100 for the pending medical imaging examinations.

The creation of the detailed attenuation value map then takes place for the limited body region 102 of the patient 101, which in the present example embodiment is formed by an organ of the patient 101 comprising the lung region. To this end, in a first method step 10 first attenuation value data from a first attenuation value measurement of the limited body region 102, in particular the lung region, of the patient 101 is firstly acquired. The acquisition of the first attenuation value data is performed by way of the magnetic resonance imaging device 140 of the medical imaging device 100, in which case the first attenuation value data is formed by first magnetic resonance data.

In a further method step 11 at least one mean attenuation value is ascertained from the first attenuation value data by means of a system control unit 105 of the medical imaging device 100 for the limited body region 102, in particular for the lung region, of the patient 101. The at least one mean attenuation value corresponds to a common attenuation value and/or a consistent attenuation value for the limited body region 102, in particular the lung region, of the patient 101. On the basis of the first attenuation value data a coarse segmentation of the body of the patient 101 moreover takes place, wherein a single mean attenuation value is associated with each segment and/or each body region. For example, the lung region of the patient 101 is acquired as a segment having a consistent mean attenuation value.

If necessary, the first attenuation value data together with the mean attenuation values are stored and/or registered by the system control unit 105 inside a storage unit 106 of the system control unit 105.

For the PET examination of the lung region and/or further organs and/or further body regions of the patient 101 which exhibit a particularly inhomogeneous density distribution and/or the density distribution of which depends on a position and/or a state of the patient 101, for example an inhaled state or an exhaled state, the mean attenuation value is too imprecise for a correct calculation of the attenuation of the PET signals. Therefore, in a further method step 12 a further acquisition takes place of second attenuation value data from a second attenuation value measurement of the limited body region 102 of the patient 101 by way of the magnetic resonance imaging device 140 of the medical imaging device 100. The second attenuation value data is also formed by magnetic resonance data in this case. In the present example embodiment the limited body region 102 of the patient 101 comprises an organ region of the patient 101 formed by the lung region.

The acquisition of the second attenuation value data takes place in this case by way of a proton density weighted attenuation value measurement by way of the magnetic resonance imaging device 140. The acquisition of the second attenuation value data in particular of the lung region of the patient 101 preferably takes place by means of a sequence which has a short or an ultrashort echo time. By preference, the echo time in this case is less than 500 μs. For the acquisition of the second attenuation value data, for example a UTE sequence and/or a PETRA sequence and/or a zTE sequence etc. is used for the magnetic resonance measurement.

If necessary, the second attenuation value data is stored and/or registered by the system control unit 105 inside the storage unit 106.

In a method step 13 following the method step 12 for acquisition of second attenuation value data, local correction values are ascertained on the basis of the second attenuation value data for the limited body region 102, in particular the lung region, of the patient 101 by way of the system control unit 105. The local correction values comprise local density values in the present example embodiment. In order to calculate and/or ascertain the local density values a segmentation of the limited body region 102 into at least two individual segments and/or subregions firstly takes place, preferably into more than two segments and/or more than two subregions. By particular preference the segmentation into at least two or more individual segments and/or at least two or more individual subregions takes place in each case on a body region with which precisely one mean attenuation value is associated. For each of the different segments and/or for each of the different subregions an ascertainment of a local density value takes place in this case on the basis of the second attenuation value data by means of the system control unit 105. In this situation the individual segments and/or subregions cover a smaller body region of the lung than a body region and/or a segment with which a mean attenuation value from the first attenuation value data is associated.

A selection and/or a determination of the individual segments and/or of the individual subregions preferably takes place spontaneously fashion and/or automatically by way of the system control unit 105. The selection and/or the determination of the individual segments and/or of the individual subregions can take place on the basis of a size of the segments and/or of the subregions and/or on the basis of a matter property of the segments and/or of the subregions and/or on the basis of a homogeneity of the segments and/or of the subregions and/or further selection criteria appearing meaningful to the person skilled in the art for the selection and/or determination of the individual segments and/or subregions.

In order to determine the local density values from the second attenuation value data, image data which shows the limited body region 102, in particular the lung region, of the patient 101 is reconstructed from the second attenuation value data by the system control unit 105. In this situation individual image elements of the reconstructed image data exhibit different gray-scale values which represent different properties of the limited body region 102, in particular of the lung region. An association of local density values with the different gray-scale values takes place on the basis of a characteristic curve, in particular on the basis of a linear characteristic curve, by way of the system control unit 105.

Parameters and/or information items from the characteristic curve are ascertained and/or created here at least partially on the basis of correlating comparison measurements which are stored within the storage unit 106 of the system control unit 105. The correlating comparison measurement can be formed for example from a correlating computed tomography measurement (CT measurement) having a suitable normalization. In addition, data from a plurality of correlating comparison measurements, in particular CT measurements, can also be stored within the storage unit 106, which means that a large region can be covered by the different comparison measurements. For example, the system control unit 105 can here determine the characteristic curve from the data from a plurality of CT measurements.

Alternatively or in addition, the characteristic curve can also be ascertained and/or created at least partially on the basis of a calibration measurement by the system control unit 105. The calibration measurement comprises in particular a measurement for the purpose of density data acquisition and/or attenuation value acquisition, in which case the calibration measurement takes place on an organ and/or on a body part of the patient having small density fluctuations and/or having small fluctuations of attenuation values by way of the magnetic resonance imaging device 140. By preference, the calibration measurement takes place on a further body region which is different to the limited body region 102, in the present instance different to the lung, of the patient 101. The further body region preferably comprises a body region of the patient 101 having an essentially homogeneous density distribution, which means that small fluctuations and/or a small variance are present in the attenuation values. For example, the further body region comprises the liver of the patient 101.

In particular, during the ascertainment and/or creation of the detailed attenuation value map of the lung region of the patient 101 the acquisition of the second attenuation value data takes place in the method step 12 depending on a first position and/or a first state of the lung. For example, the acquisition of the second attenuation value data takes place only in an inhaled state and/or in an exhaled state of the patient 101. This is in particular advantageous if the PET examination is also carried out in the first position of the patient 101 as a so-called “gated-PET examination”.

In addition, local density values for a second position of the lung and/or of the lung region of the patient 101 can be calculated and/or determined by the system control unit 105, in which case to this end the second attenuation value data for the first position of the lung and/or of the lung region and/or the local density values for the first position of the lung and/or of the lung region are incorporated into the calculation for the local density values of the second position. The calculation of the local density values in the second position of the lung and/or of the lung region of the patient 101 is carried out by the system control unit 105 on the assumption that a total mass and/or a total volume of the material to be attenuated, for example a total volume of bodily fluids, such as blood or water, and/or a total volume of tissue, within the limited body region 102, in particular within the lung region, of the patient 101 in both positions and/or in both states of the limited body region, in particular of the lung region, in the different positions and/or states of the lung, is equal.

Furthermore, in the method step 13 for ascertaining the local density values for the limited body region 102, in particular the lung region, of the patient 101 a gravitational force acting on the lung region is taken into consideration. By means of the gravitational force a density gradient arises in the limited body region 102, in which case a density increases inside the lung region along the gravitational force. The density gradient can be determined on the basis of the second attenuation value data by way of the system control unit 105 and/or read out from a database. Incomplete measurements, for example magnetic resonance measurements with only one or a few two-dimensional layers of the lung region, can also be completed by the system control unit 105 by means of the density gradient.

The method steps 12, 13 for the acquisition of second attenuation value data and the ascertainment of local density values on the basis of the second attenuation value data for the limited body region 102, in particular the lung region, of the patient 101 can take place at least partially simultaneously and/or in temporal succession with respect to the method steps 10, 11 of the acquisition of first attenuation value data and of the ascertainment of the mean attenuation value data.

After the method step 11 of the ascertainment of the mean attenuation value for the limited body region 102, in particular the lung region, of the patient 101 and the method step 13 of the ascertainment of the local density values for the limited body region 102, in particular the lung region, of the patient 101, a determination of the detailed attenuation value map for the lung region of the patient 101 takes place by means of the system control unit 105 in a further method step 14. Both the mean attenuation values and also the local density values are incorporated into the detailed attenuation value map of the lung of the patient 101. Material properties and/or attenuation properties are determined for the individual subregions and/or the individual segments by the system control unit 105 on the basis of the local density values and/or the local proton density of subregions and/or segments of the limited body region 102 of the patient 101. In this situation the mean attenuation value is scaled and/or corrected by the system control unit 101 by means of the local density values and/or the material properties and/or the attenuation properties for each segment and/or each subregion. In such a manner attenuation values in the thorax region, in particular in the lung region, of the patient 101 are made available with a high degree of accuracy.

The detailed attenuation value map is ascertained and/or made available by the system control unit 105, in which case the detailed attenuation value map is firstly ascertained and/or made available on the basis of the local density values for the first position and/or the first state of the limited body region 102, in particular of the lung region. Alternatively or in addition, a detailed attenuation value map can also be ascertained and/or made available by the system control unit 105 for the second position and/or the second state of the limited body region 102 formed by the lung region. Incorporated into the calculation of the detailed attenuation value map for the second position of the limited body region 102, in particular of the lung region, are local density values for the second position of the limited body region 102, in which case the local density values for the second position were ascertained on the basis of the second attenuation value data for the first position of the limited body region 102, in particular of the lung region.

The magnetic resonance imaging device 140 of the medical imaging device 100 is illustrated schematically in FIG. 1 and comprises a magnet unit 141. The magnet unit 141 surrounds a patient receiving area 103 for accommodating the patient 101, in which case the patient receiving area 103 is enclosed in a cylindrical fashion in a circumferential direction by the magnet unit 141. The patient 101 can be slid into the patient receiving area 103 by means of the patient supporting device 104 of the medical imaging device 100. To this end the patient supporting device 104 is arranged to be capable of movement inside the patient receiving area 103.

The magnet unit 141 comprises a primary magnet 142 which is designed for generating a strong and in particular constant primary magnetic field 143 during operation of the magnetic resonance imaging device 140. The magnet unit 141 furthermore has a gradient coil unit 144 for generating magnetic field gradients which are used for position encoding during an imaging operation. Furthermore, the magnet unit 141 comprises a high-frequency antenna unit 145 which is designed in order to excite polarization which arises in the primary magnetic field 143 generated by the primary magnet 142.

In order to control the primary magnet 142 of the gradient coil unit 144 and to control the high-frequency antenna unit 145 the magnetic resonance imaging device 140 has a control unit 146 formed by a central processing unit. The control unit 146 centrally controls the magnetic resonance imaging device 140, such as for example the execution of a predetermined imaging gradient echo sequence. To this end the control unit 146 comprises a gradient control unit (not illustrated in detail) and a high-frequency antenna control unit (not illustrated in detail). The control unit 146 furthermore comprises an evaluation unit for the evaluation of magnetic resonance image data.

The PET unit 120 of the medical imaging device 100 comprises a plurality of positron emission tomography detector elements (PET detector elements) which are arranged to form the PET detector array 121. The PET detector array 121 comprises a scintillation detector array having scintillation crystals, for example LSO crystals. The PET unit 120 furthermore comprises a photodiode array, for example an avalanche photodiode array or APD photodiode array, which is arranged downstream of the scintillation detector array inside the PET unit 120. The PET detector array 121 furthermore has a detector electronics unit (not illustrated in detail) which comprises an electrical amplifier circuit and further electronic components (not illustrated in detail). In order to control the detector electronics unit and the PET detector array 121 the PET unit 120 has a control unit 122 formed by a central processing unit. The control unit 122 centrally controls the PET unit 120. The control unit 122 furthermore comprises an evaluation unit for the evaluation of PET data.

The medical imaging device 100 furthermore has the central system control unit 105 which for example coordinates the acquisition of magnetic resonance image data and of PET image data. The system control unit 105 furthermore comprises an evaluation unit (not shown in detail). The system control unit 105 moreover comprises a computer program product which comprises a program and can be loaded directly in the storage unit 106 of the programmable system control unit 105 of the medical imaging device 100. The computer program product comprises program segments/modules and/or software which are designed to execute the described method for creating the detailed attenuation value map together with the magnetic resonance imaging device 140 and the PET unit 120 when the program is executed in the system control unit 105 of the medical imaging device 100. Furthermore, the system control unit 105 can have further programs and/or further software which are necessary for execution of the method for creating the detailed attenuation value map and/or for operation of the medical imaging device 100.

Control information, such as for example imaging parameters as well as reconstructed image data, can be displayed on a display unit 107, for example on at least one monitor, of the medical imaging device 100 for an operator. The medical imaging device 100 moreover has an input unit 108 by means of which information and/or parameters can be entered by an operator during a measurement operation.

The medical imaging device 100 presented can naturally comprise further components which medical imaging devices normally have. A general mode of operation of a medical imaging device 100 is furthermore known to the person skilled in the art, which means that a detailed description of the general components is dispensed with.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in detail on the basis of the preferred example embodiment, the invention is not limited by the disclosed examples and other variations can be derived herefrom by the person skilled in the art, without departing from the scope of protection of the invention.

Although the invention has been illustrated and described in detail by way of the preferred example embodiment, the invention is not restricted by the disclosed examples and other variations can be derived therefrom by the person skilled in the art without departing from the scope of protection of the invention.

What is claimed is:

1. A method for creating a detailed attenuation value map for a limited body region of a patient for a positron emission tomography examination by way of a medical imaging device, comprising:
acquiring first attenuation value data from a first attenuation value measurement of the limited body region via the medical imaging device;
ascertaining at least one mean attenuation value based upon the first attenuation value data for the limited body region;
acquiring second attenuation value data from a second attenuation value measurement of the limited body region via the medical imaging device;
ascertaining local correction values based upon the second attenuation value data for the limited body region; and
determining the detailed attenuation value map for the limited body region based upon the at least one mean attenuation value and the local correction values.

2. The method of claim 1, wherein the acquiring of the second attenuation value data takes place based upon a proton density weighted attenuation value measurement, in which the local correction values comprise local density values.

3. The method of claim 1, wherein the limited body region comprises an organ region of the patient.

4. The method of claim 3, wherein the organ region comprises a lung region of the patient.

5. The method of claim 1, wherein, based upon the second attenuation value data, a segmentation of the limited body region takes place and a local correction value is ascertained in each case for different segments of the limited body region based upon the second attenuation value data.

6. The method of claim 1, wherein image data is reconstructed based upon the second attenuation value data and the local correction value is determined at least partially by way of an association of correction values with the reconstructed image data based upon a characteristic curve.

7. The method of claim 6, wherein at least one of at least one parameter and at least one information item from the characteristic curve is determined for an association of the second attenuation value data with a local correction value based upon at least one of correlating comparison measurements and a calibration measurement on a further body region, which is different from the limited body region, of the patient.

8. The method of claim 1, wherein a gravitational force acting on the limited body region is incorporated into the calculation of the local correction values.

9. The method of claim 1, wherein the acquisition of the second attenuation value data takes place by way of at least one sequence having a short echo time.

10. The method of claim 1, wherein the detailed attenuation value map is created depending on a first position of the limited body region.

11. The method of claim 10, wherein the detailed attenuation value map for a second position of the limited body region is calculated based upon the second attenuation value data for the first position of the limited body region.

12. A medical imaging device comprising:
a positron emission tomography unit;
a magnetic resonance imaging device; and
a system control unit configured to create a detailed attenuation value map for a limited body region of a patient for a positron emission tomography examination, and configured to:
acquire first attenuation value data from a first attenuation value measurement of the limited body region by the magnetic resonance imaging device,
ascertain at least one mean attenuation value based upon the first attenuation value data for the limited body region,
acquire second attenuation value data from a second attenuation value measurement of the limited body region by the magnetic resonance imaging device,
ascertain local correction values for the limited body region based upon the second attenuation value data, and
determine the detailed attenuation value map for the limited body region based upon the at least one mean attenuation value and the local correction values.

13. A computer program product comprising a program, loadable directly in a storage unit of a programmable system control unit of a medical imaging device, and including program segments to execute the method of claim 1 when the program is executed in the system control unit of the medical imaging device.

14. The method of claim 2, wherein the limited body region comprises an organ region of the patient.

15. The method of claim 14, wherein the organ region comprises a lung region of the patient.

16. The method of claim 2, wherein the acquisition of the second attenuation value data takes place by way of at least one sequence having a short echo time.

17. The method of claim 2, wherein the detailed attenuation value map is created depending on a first position of the limited body region.

18. A computer program product comprising a program, loadable directly in a storage unit of a programmable system control unit of a medical imaging device, and including program segments to execute the method of claim 2 when the program is executed in the system control unit of the medical imaging device.

19. A computer program product comprising a program, loadable directly in a storage unit of a programmable system control unit of a medical imaging device, and including program segments to execute the method of claim 3 when the program is executed in the system control unit of the medical imaging device.
